(19) United States
(12) Patent Application Publication
(10) Pub. No.: US 2026/0272345 A1
(43) Pub. Date: Sep. 17, 2026

(54) METHOD AND SYSTEM FOR DETECTION OF COGNITIVE IMPAIRMENT

(71) Applicant: UNIVERSITY OF HOUSTON SYSTEM, Houston, TX (US)

(72) Inventors: Stacey GORNIAK, Houston, TX (US); Luca POLLONINI, Manvel, TX (US); Arturo E. HERNANDEZ, Manvel, TX (US)

(73) Assignee: UNIVERSITY OF HOUSTON SYSTEM, Houston, TX (US)

(21) Appl. No.: 19/472,136

(22) PCT Filed: Apr. 17, 2024
(86) PCT No.: PCT/US2024/025025

§ 371 Date: Oct. 3, 2025

Related U.S. Application Data
(60) Provisional application No. 63/460,237, filed on Apr. 18, 2023.

(51) Int. Cl. A61B 5/1455; A61B 5/00; A61B 5/145

(52) U.S. Cl. CPC A61B 5/14553; A61B 5/14546; A61B 5/14552; A61B 5/4088; A61B 5/6814; A61B 5/6839; A61B 5/7246; A61B 5/7278; A61B 5/742; A61B 5/748; A61B 2562/0238

(57) ABSTRACT

A system and method to detect cognitive impairment is disclosed. A head mounted sensor is operable to be worn by a user. The head mounted sensor includes an optical sensor for collecting an optical response from a region of interest in the brain of the user. A user input interface receives a user test input from a test device executing a stimulation test to the user. A controller is coupled to the sensor device and the user input interface. The controller collects optical response data from the optical sensor and test input data from the test device. The controller determines a hemoglobin level in the region of interest based on the optical response data. The determined hemoglobin level in the region of interest is correlated with the user test input in response to the stimulation test. Brain activity is evaluated based on the determined hemoglobin level.

### PRIORITY CLAIM

The present disclosure claims the benefit of and priority to U.S. Provisional Application Ser. No. 63/460,237 filed Apr. 18, 2023. The contents of that application are hereby incorporated by reference in their entirety.

### TECHNICAL FIELD

The present invention relates generally to detection of cognitive impairment. More specifically, methods and systems for detection of hemoglobin levels via a blade-shaped headgear including optical sensors for purposes of determining brain activity in response to stimulus for determining cognitive decline are disclosed.

### BACKGROUND

Determination of changes in areas of the brain is valuable to determine cognitive impairment for numerous applications for disease treatment. For example, changes in the chemical state of selected areas of the brain may be used for detection of early stages of dementia (known as mild cognitive impairment, including amnesiac mild cognitive impairment).

Another example of the need for detection of cognitive impairment is for patients with diabetes. Nearly 24% of the 40 million individuals in the United States over the age of 60 are currently living with type II diabetes mellitus (DM). Persons with DM (PwDM) experience decline in hand/finger sensorimotor function as compared to healthy individuals; however, self-awareness of these changes is low. Reduced functional hand use has been associated with a loss of independent living and reduced quality of life in PwDM. Tactile dysfunction due to peripheral neuropathy (PN) has been implicated as the primary cause of motor deficits in PwDM. Motor changes in PwDM occur independent of tactile impairment, unrelated to disease duration and severity. Altered hemodynamic responses due to both micro-and macrovascular changes have been implicated as a potential source of global motor changes in PwDM.

Specifically, endothelial dysfunction within dermal and muscle tissues in adult PwDM has been related to altered motor behaviors, including abnormal force production. Altered hemodynamic function of the cortex occurs during tactile sensory and manual motor tasks. Further, these altered hemodynamic patterns directly relate to poor tactile detection thresholds and motor performance in PwDM.

A current method to detect changed cortex function is use of functional magnetic resonance imaging (fMRI). However, fMRI limits the possible participants, as implanted devices (e.g., stents, pacemakers, etc.) commonly used to treat cardiovascular comorbidities of DM are an exclusion criterion for using fMRI. fMRI also fails to determine critical changes in regions of interest of the brain in response to stimulus.

Recently, it has been determined that changes in cortical oxygenation indices, such as oxygenated hemoglobin (HbO) and deoxygenated or reduced hemoglobin (HbR) occur in patients during performance of tactile sensory and manual motor tasks. For example, cortical hemodynamic activity may be measured with functional near-infrared spectroscopy (fNIRS) alongside individual task performance with the goals of (1) detecting differences between PwDM and controls and (2) evaluating the relationship between cortical hemodynamic activity and task performance. However, currently it is difficult to measure such changes as traditional neuroimaging approaches do not show relevant cortical activity.

There is a need for a readily accessible instrument that detects changes in oxygenation indices that occur with performance of tasks to detect disorders such as dementia. There is a further need for a detection device that is safe for use in populations with metal implants, is cost-effective, and can be used in ambulatory settings. There is a need for a detection system that allows tracking the progress of dementia and functional changes in parts of the brain that are impacted by early stages of dementia.

### SUMMARY

One disclosed example is a system to evaluate brain activity. The system includes a head mounted sensor operable to be worn by a user. The head mounted sensor includes an optical sensor for measuring an optical response from a region of interest in the brain of the user. A user input interface receives a user test input from a test device executing a stimulation test to the user. A controller is coupled to the sensor device and the user input interface. The controller collects optical response data from the optical sensor and user test input from the user input interface. The controller is operable to determine a hemoglobin level in the region of interest based on the optical response data. The controller correlates the determined hemoglobin level in the region of interest with the user test input in response to the stimulation test. The controller evaluates brain activity based on the determined hemoglobin level.

A further implementation of the example system is an embodiment where the head mounted sensor includes an underside. The underside includes teeth to interface with the hair of the user to stabilize the head mounted sensor when worn by the user. Another implementation is where the head mounted sensor includes a curved support structure conforming to the head of the user and lateral wings coupled to the support structure. Another implementation is where the optical sensor is positioned in proximity to the region of interest of the brain of the user when the sensor is worn by the user. Another implementation is where the region of interest of the brain is at least one of an anterior pre-frontal cortex (aPFC); a dorsolateral pre-frontal cortex (dPFC); a supplemental motor area (SMA); a primary motor cortex (M1); a primary sensory cortex; a visuomotor coordination area; or a supramarginal gyrus. Another implementation is where the input interface is in communication to a mobile device administering the stimulation test to the user, wherein the mobile device includes a transceiver to send the input data to the controller. Another implementation is where the mobile device executes an application to administer the stimulation test, and wherein the stimulation test is one of a working memory (N-back) evaluation or the Montreal Cognitive Assessment (MoCA). Another implementation is where the stimulation test is one of a combined memory test and motor function test or a memory test. Another implementation is where the system includes a display coupled to the controller. The display displays the brain activity of the region of interest of the brain of the user based on the collected optical data and an indication of cognitive impairment based on the brain activity. Another implementation is where the cognitive impairment is dementia. Another implementation is where the optical sensor is an optode that includes a dual-wavelength LED emitting light to the region of interest, a silicon photodiode and an optical fiber coupled to the silicon photodiode. The silicon photodiode collects backscattered light from the region of interest and transmits the signal to the controller via the optical fiber. Another implementation is where the optical response is associated with concentration changes of one of oxygenated (HbO) hemoglobin, deoxygenated hemoglobin (HbR), total hemoglobin, or glycated hemoglobin in the region of interest.

Another disclosed example is a method of evaluating brain activity in a user. A user test input is received from a test device executing a stimulation test to the user; An optical input signal is provided to a region of interest of the brain of the user via a head mounted sensor worn by the user. Optical response data is collected from an optical detector in the region of interest. A hemoglobin level is determined in the region of interest based on the optical response data. The determined hemoglobin level in the region of interest is correlated with the user test input in response to the stimulation test. Brain activity is evaluated based on the determined hemoglobin level.

A further implementation of the example method is an embodiment where the region of interest of the brain is at least one of an anterior pre-frontal cortex (aPFC); a dorsolateral pre-frontal cortex (dPFC); a supplemental motor area (SMA); a primary motor cortex (M1); a primary sensory cortex; a visuomotor coordination area; or a supramarginal gyrus. Another implementation is where the stimulation test includes one of a working memory (N-back) evaluation, the Montreal Cognitive Assessment (MoCA), a combined memory test and motor function test, or a memory test. Another implementation is where the method includes displaying brain activity of the region of interest of the brain of the user based on the collected optical data and an indication of cognitive impairment based on the brain activity. Another implementation is where the method includes determining cognitive impairment from the evaluation of brain activity. Another implementation is where the optical signal is provided via an optical emitter including a dual-wavelength LED emitting light to the region of interest. The optical detector is a silicon photodiode and an optical fiber coupled to the silicon photodiode. The silicon photodiode collects backscattered light from the region of interest. Another implementation is where the optical response is associated with concentration changes of one of oxygenated (HbO) hemoglobin, deoxygenated hemoglobin (HbR), total hemoglobin, or glycated hemoglobin in the region of interest. Another implementation is where the head mounted sensor includes an underside. The underside includes teeth to interface with the hair of the user to stabilize the head mounted sensor when worn by the user. Another implementation is where the head mounted sensor further includes a curved support structure conforming to the head of the user and lateral wings coupled to the support structure.

Another disclosed example is a head mounted device for collecting data for evaluating brain activity in a patient. The device includes a blade structure having a curved shape operable for contact between the forehead and back of the head of a patient. The device includes a series of optodes in the blade structure. The optodes each include a dual-wavelength LED, a silicon photodiode and an optical fiber. Each of the optodes are located in proximity to a respective region of interest of the brain of the patient indicative of brain activity. The LED emits an input signal to the respective region of interest and the silicon photodiode collects a response optical signal from the respective region of interest. A data interface collects the response optical signals from the optical fibers of the optodes to determine hemoglobin levels of the respective regions of interest.

A further implementation of the example head mounted device is an embodiment where the blade structure includes an underside configured to contact the head of the patient, the underside including teeth attaching to hair of the patient. Another implementation is where the head mounted device includes a transceiver to send the collected optical signals to an external device. Another implementation is where the head mounted device includes a pair of lateral wings extending perpendicularly from the blade structure. The lateral wings contact the head of the patient to hold the head mounted device in place.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of embodiments together with reference to the accompanying drawings.

FIG. 1 shows a block diagram of an example cortical hemodynamic evaluation system including a wearable functional near-infrared spectroscopy sensor device;

FIG. 2 is a perspective view of the wearable sensor device in FIG. 1;

FIG. 3A is a side view of the wearable sensor device in FIG. 2 worn by a user;

FIG. 3B is a front view of the wearable sensor device in FIG. 2 worn by a user;

FIG. 4A is a block diagram of one of the optodes in the wearable sensor device in FIG. 2 in relation to the brain of the user;

FIG. 4B is a block diagram of an alternate method of deploying one of the optodes of the wearable sensor device in FIG. 2 in relation to the brain of the user;

FIG. 5A shows a side view of areas of interest of the brain in relation to the example wearable sensor device;

FIG. 5B shows a side view of the brain and areas of interest;

FIG. 5C shows a top view of the cortical layout of the optode optical emitters and detectors of the example wearable sensor device on the brain relative to areas of interest;

FIG. 5D shows a sensitivity map overlaid onto a brain model for areas of interest sensed by the example wearable sensor device;

FIG. 6 shows a flow diagram of the process of testing a patient using the example wearable device and analysis system;

FIG. 7A shows an example patient input interface for a clinician to obtain patient information for a patient being evaluated by the example cortical hemodynamic evaluation system;

FIG. 7B shows another example interface for data from performance of a memory based task from the example cortical hemodynamic evaluation system;

FIG. 7C shows another example interface for data from performance of a behavior based task from the example cortical hemodynamic evaluation system;

FIG. 8A shows an output interface that shows test results of a patient from the example cortical hemodynamic evaluation system in FIG. 1;

FIG. 8B shows another output interface that shows mapping of areas of interest in the brain and cognitive analysis from the example cortical hemodynamic evaluation system in FIG. 1;

FIG. 9A shows an example output interface generated by the example cortical hemodynamic evaluation system in FIG. 1 that indicates a positive outcome for MCI detection;

FIG. 9B shows an example output interface generated by the example cortical hemodynamic evaluation system in FIG. 1 that indicates a negative outcome for MCI detection;

FIG. 10 is a table showing the demographics for participants in experiments testing the example the example cortical hemodynamic evaluation system in FIG. 1;

FIG. 11 shows the sequence of images for the single task and dual task testing for the experiments;

FIG. 12 shows a series of graphs of the results of the MoCA tests by the participants in the experiments;

FIG. 13A shows graphs of the results of tests of the example cortical hemodynamic evaluation system involving performing a single task;

FIG. 13B shows a graph of the results of tests of the example cortical hemodynamic evaluation system involving performing a dual task;

FIG. 13C shows a graph of single task differences in hemoglobin across N-back condition in tests of the example cortical hemodynamic evaluation system; and

FIG. 14 shows a graph of the t-scores of hemoglobin in different regions of interest in the tests of the example cortical hemodynamic evaluation system.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to an example cortical hemodynamic evaluation system that allows determination of cognitive decline. The example cortical hemodynamic evaluation includes a head mounted sensor device system that monitors brain activity of a user in response to performing cognitive based test activities. The system allows detection of early stages of dementia (known as mild cognitive impairment (MCI), including amnesiac mild cognitive impairment) using a wearable device and associated software. The example system may also track the progress of dementia. The example system detects functional changes in parts of the brain that are impacted by early stages of dementia via functional near infrared spectroscopy sensors. The example system includes a wearable sensing device that utilizes functional near infrared spectroscopy to measure cortical activity that is not detected using traditional neuroimaging approaches. The example system device is safe for use in populations with metal implants, is cost-effective, and can be used in ambulatory settings.

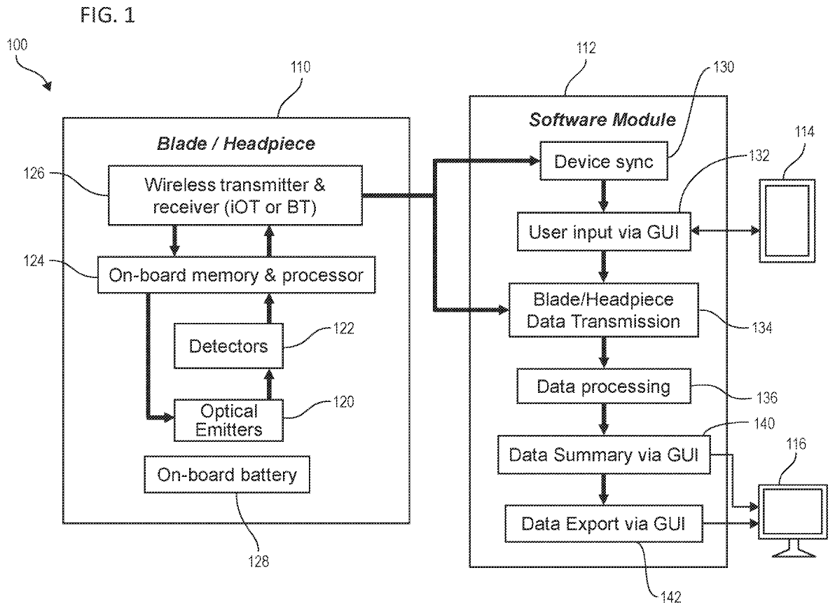

FIG. 1 shows a block diagram of an example cortical hemodynamic evaluation system 100 that tests patients for cognitive decline such as the onset of dementia or other disorders based on brain activity. The system 100 includes a wearable headpiece sensor device 110 and an analysis system 112. The analysis system 112 may be coupled to a mobile device 114 operated by the user as well as a workstation display 116. As will be explained the workstation display 116 may display control interfaces and output interfaces generated by the analysis system 112 that may be viewed by a clinician or other health care professional. The interfaces displayed on the display 116 allow the clinician to enter data and control the operation of the analysis system 112 as will be explained below.

The headpiece device 110 includes a head mounted sensor structure operable to be worn by a user. The headpiece device 110 is based on functional near infrared spectroscopy analysis. The headpiece device 110 includes a series of optical sensors or optodes that include optical emitters 120 and a series of detectors 122, a control module 124 having on-board memory and a controller, and a wireless transceiver 126. The components of the headpiece device 110 are powered by an onboard battery 128. The headpiece sensor device 110 may be rechargeable through a standard port such as a USB port.

In this example, the transceiver 126 maybe a IOT or Bluetooth compatible device. The transceiver 126 allows detected optical data from the detectors 122 of the optical sensors to be sent to the analysis system 112. In this example, detected optical data may also be transmitted from the wearable sensor device 110 through a wire connection such as the USB port. The optical emitters 120 and detectors 122 serve as optical sensors termed optodes for collecting an optical response from different regions of interest in the brain of the user.

The analysis system 112 includes a device synchronization module 130, a user input interface 132, a wearable device data transmission interface 134, and a data processing module 136. An internal display may include a user interface that allows data to be summarized and exported from the analysis system 112 to an external device for further analysis.

The device synchronization module 130 synchronizes data collection from the optodes of the wearable device 110 and the inputs that respond to the test performed by the user on the test device. In this example, a user input interface 132 is in communication with the mobile device 114 serves as a test device for executing cognitive tests such as a stimulation test to the user. The input data from the user in response to the test may be entered on the display of the mobile device 114. The mobile device 114 includes a transceiver to send the input data to the data processing module 136. The analysis system 112 receives user inputs at the user interface 132 from the mobile device 114 when the user takes the test executed by the mobile device 114. The data processing module 136 incudes a controller that collects optical response data from the optical sensors of the headpiece device 110 when the user is taking the stimulation test.

The data transmission interface 134 receives data from the wearable sensor device 110. In this example, once data is collected on the user input from responding to the simulation test and the corresponding optical response data from the wearable sensor device 110, the controller of the data processing module 136 determines a hemoglobin level in the region of interest based on the optical response data from the optodes. The controller correlates the determined hemoglobin level in the region of interest with the user test input in response to the stimulation test. The controller of the data processing module 136 evaluates brain activity based on the determined hemoglobin level. The brain activity is correlated with the test inputs to detect signs of cognitive impairment. As will be explained the data processing module 136 may generate a series of data summaries via a data summary output module 140 that may display data summaries on the display 116. The data processing module 136 may also generate data export via a data export module 142 that allows display of exported data on the display 116.

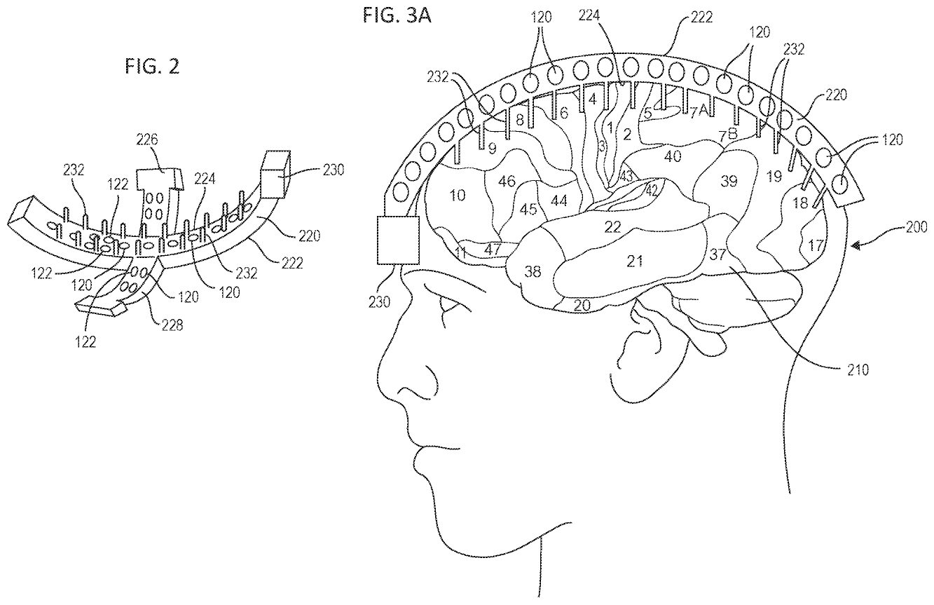

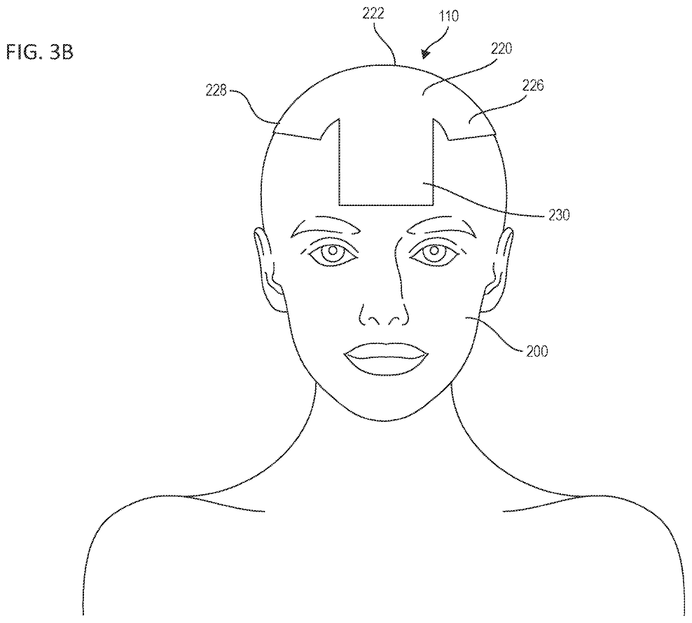

FIG. 2 is a perspective view of the wearable sensor device 110. FIG. 3A is a side view of the wearable sensor device 110 worn on the head of a user 200. FIG. 3B is a front view of the wearable sensor device 110 worn by the user 200. Regions of the brain 210 of the user 200 are shown in FIGS. 3A-3B.

As shown in FIG. 2, the wearable sensor device 110 has a curved blade shaped main body support structure 220 that includes an outside surface 222 and an underside curved surface 224. The body support structure 220 is thus shaped to be worn on the scalp of the head of a user. The interior of the main body 220 holds the support components of the wearable sensor device 110 such as electronics, external interfaces, a battery, and the like detailed in FIG. 1. The main body 220 is attached to parallel lateral wings 226 and 228 that extend laterally from the main body 220. The lateral wings 226 and 228 assist in holding the main body support structure 220 to the head of the user 200. A series of optical emitters 120 and optical detectors 122 are positioned on the underside curved surface 224. The optical emitters 120 and optical detectors 122 are also positioned on the underside of the lateral wings 226 and 228. The positioning of the optical emitters 120 and detectors 122 on the underside curved surface 224 are based on proximity to areas of interest in the brain of the user 200 when the wearable sensor device 110 is worn by the user. When the wearable sensor device 110 is worn, additional respective optical emitters and optical detectors on the underside of the lateral wings 226 and 228 may be positioned in proximity to regions of interest near the sides of a head.

The underside curved surface 224 includes teeth 232 that mesh with the hair of the user to stabilize the head mounted wearable sensor device 110. The teeth 232 that interface with the hair and scalp of the head of the user help keep the head mounted sensor 110 stable during data collection. A front end 230 of the main support structure 220 may serve as a forehead contact. The front end 230 may be attached to the user via tape or adhesive to assist in stabilizing the head mounted wearable sensor device 110 on the head of the user 200.

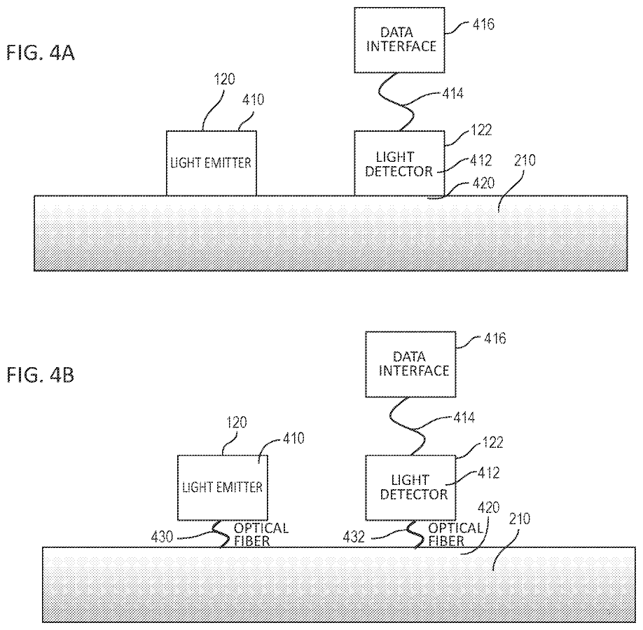

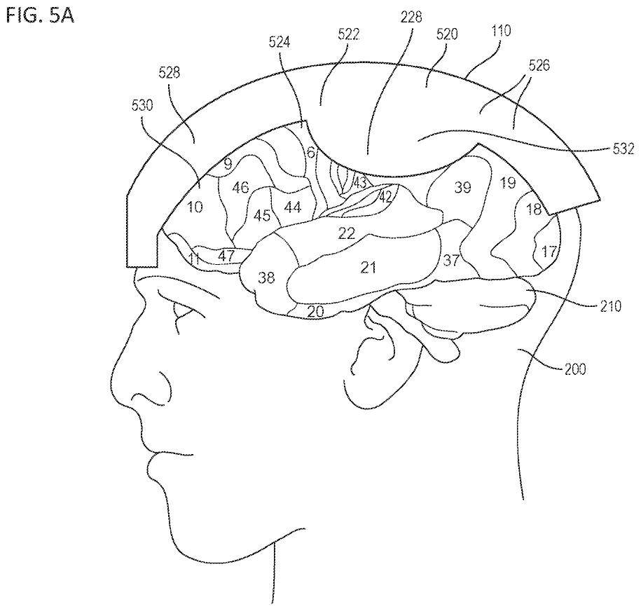

FIG. 4A is a block diagram of one of the optodes in the wearable device sensor in FIG. 2 in relation to the brain 210 of the user. The optodes include an optical emitter 120 that are each a light emitter emitting at least two wavelengths. Such a light emitter may be a dual-wavelength LED, two adjacent single-wavelength LEDs, two single wavelength lasers, or a more-than-two-wavelengths LEDs or lasers or the like. In this example, each of the optodes include a light emitter 410 that is a dual-wavelength LED that emits light to the region of interest on the head of the user. In this example, the detectors 122 each include a silicon photodiode 412, and an optical fiber 414 coupled to the silicon photodiode 412. Alternatively, the detector 122 may be an avalanche photodiode, a photomultiplier, or the like. In this example, the light emitter 410 and the silicon photodiode 412 both are in direct contact with a head surface 420 of the user. Light is emitted by the optode through the region of interest on the head surface 420. The silicon photodiode 412 collects backscattered light from the region of interest and transmits the optical signal to the controller via the optical fiber 414. The other end of the optical fiber 414 is thus coupled to an optical signal interface 416 allows the optical signal to be transmitted to the data transmission interface 134 in FIG. 1.

FIG. 4B is a block diagram of an alternate method of deploying one of the optodes including an optical emitter and detector of the wearable sensor device 110 in FIG. 2 in relation to the brain 210 of the user. In this example, the light emitter 410 is optically coupled to one end of an emitter optic fiber 430. The light emitter 410 emits light through the optic fiber 430. The other end of the emitter optic fiber 430 is in contact with the surface of the head 420 in proximity to the region of interest. Similarly, one end of a detector optic fiber 432 is in contact with the surface of the head 420. The other end of the detector optic fiber 432 is optically coupled to the silicon photodiode 412. Thus, the emitter optic fiber 430 guides the emitted light to the region of interest. The silicon photodiode 412 collects backscattered light guided through the emitter optic fiber 432 from the region of interest via the detector optic fiber 432 and transmits the via the optical fiber 414. The detector optic fiber 432 assists in guiding the backscattered light from the region of interest. The other end of the optical fiber 414 is thus coupled to an optical signal interface 416 that communicates with the data transmission interface 134 in FIG. 1.

In this example, the optical response in different regions of interest of the brain are associated with concentration changes of oxygenated (HbO) hemoglobin, deoxygenated hemoglobin (HbR), total hemoglobin, or glycated hemoglobin in the respective region of interest. As will be explained, the concentration changes in hemoglobin may be correlated with user inputs in response to the user inputs in response to the cognitive test executed by the mobile device 114 in FIG. 1.

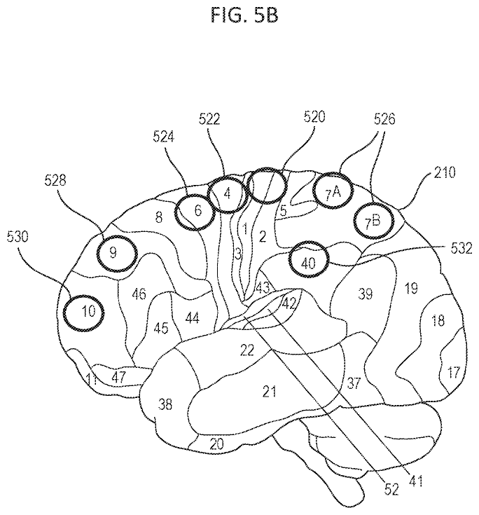

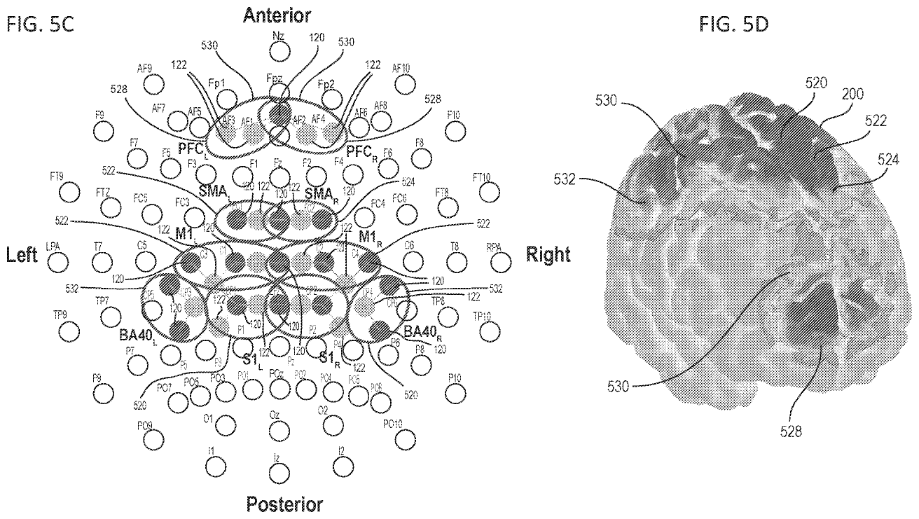

FIG. 5A shows an outline of the wearable sensor device 110 in relation to the brain 210 of a user in relation to different areas of interest. FIG. 5B shows the areas of interest on the brain 210. As explained above, concentration changes in hemoglobin at each region of interest may be measured by the wearable sensor device 110 through the optodes.

In this example, the regions of interest include the primary sensory cortex (S1) 520, the primary motor cortex (M1) 522, the supplemental motor area (SMA) 524, the visuomotor coordination area 526, the dorsolateral pre-frontal cortex (dPFC) 528, the anterior pre-frontal cortex (aPFC) 530, and the supramarginal gyrus 532.

In this example, the optical emitters 120 and detectors 122 in the underside 224 of the support 220 of the wearable sensor device 110 may monitor hemoglobin concentrations in the areas 520, 522, 524, 526, 528, and 530. The optical emitters 120 and detectors 122 in the lateral wings 226 and 228 may be used to monitor hemoglobin concentrations in the supramarginal gyrus 532. FIG. 5C is a top view of the brain 210 that shows the relative positions of the areas of interest 520, 522, 524, 526, 528, 530, and 532 and respective optical emitters 120 and detectors 122 that are positioned by the wearable sensor device 110. Multiple detectors 122 may sense the scattered light from a single optical emitter 120. Certain optodes have a single detector 122 associated with a single emitter 120. FIG. 5D is a perspective view of the brain 210 showing the areas of interest 520, 522, 524, 526, 528, 530, and 532.

In this example, the mobile device 114 may execute an application to apply a cognitive test for the user to respond to inputs shown on the display of the mobile device 114. The application may be downloaded to the mobile device 114 from a central repository such as an app store. In this example, the test may be a working memory (N-back) evaluation or the Montreal Cognitive Assessment (MoCA). Other cognitive tests may include a stimulation test such as a motor function test, a memory test, tests from the NIH Toolbox, or other test of brain function.

The controller of the data processing module 136 executes an application to convert the collected optical data from the detectors 122 on the wearable sensor device 110 to hemoglobin levels correlated with activity in the corresponding regions of interest. The application will correlate the determined hemoglobin levels with the region of interest and time periods associated with the inputs from the user taking the cognitive test executed on the mobile device 114. The controller may then generate output interfaces on the display 116 using the summary data module 140.

Various output interfaces may be displayed on the display 116. For example, one interface may display the brain activity of the region of interest of the brain of the user and an indication of cognitive impairment based on the brain activity.

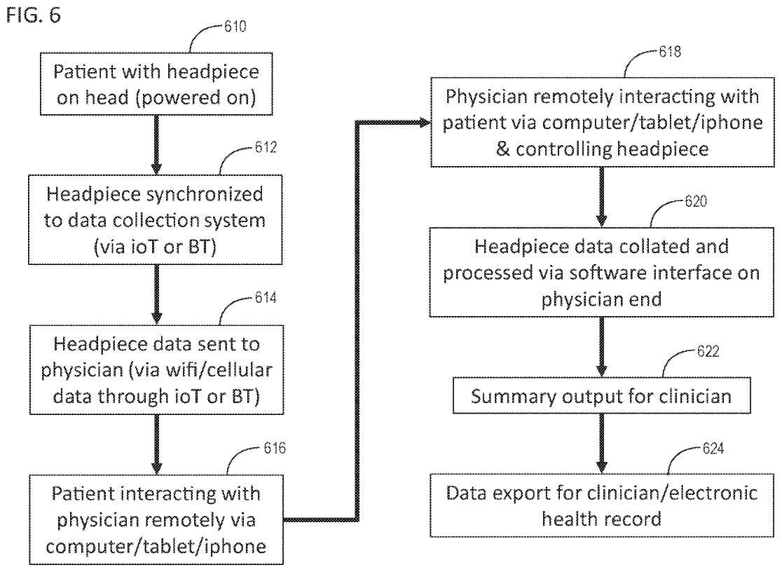

FIG. 6 shows a flow diagram of the testing process using the system 100 in FIG. 1. The patient first puts on the wearable sensor device 110 (610). The wearable sensor device 110 is powered up. The wearable sensor device 110 exchanges handshake signals with the analysis system 112 via either a wired connection or a wireless connection (612). The data collected from the optical emitters 120 and detectors 122 on the wearable sensor device 110 are sent to the analysis system 112 (614). The physician then interacts with the patient via the user device 114 to execute the cognition test (616). Such tests may be conducted under direct supervision or remotely such as via a video link between the patient and the physician. Patient related data may be collected via either the physician requesting entry through the mobile device 114 or the physician directly entering patent data through an interface on the display 116 (618). The data from the optical emitters and detectors during the test from the regions of interest are collected and processed by the controller of the data processing module 136 (620). In this example, a summary output is prepared by the data summary module 140 (622). Data export to the workstation is also performed via the data export module 142 (624).

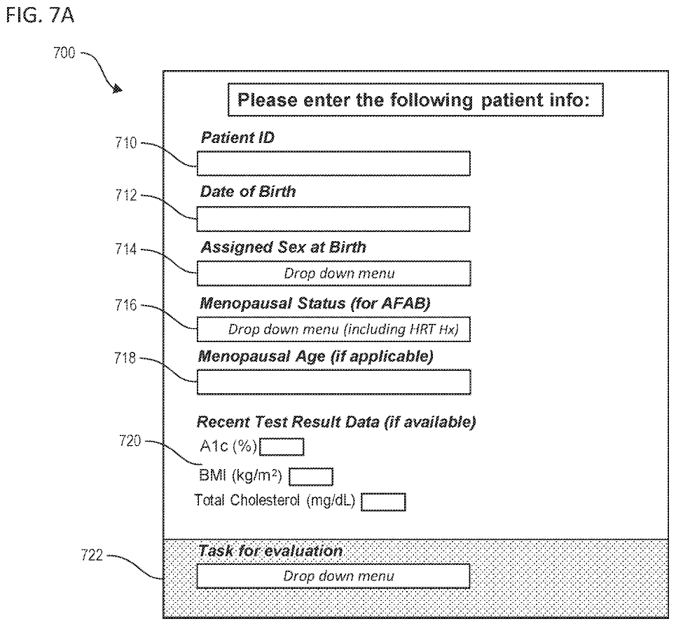

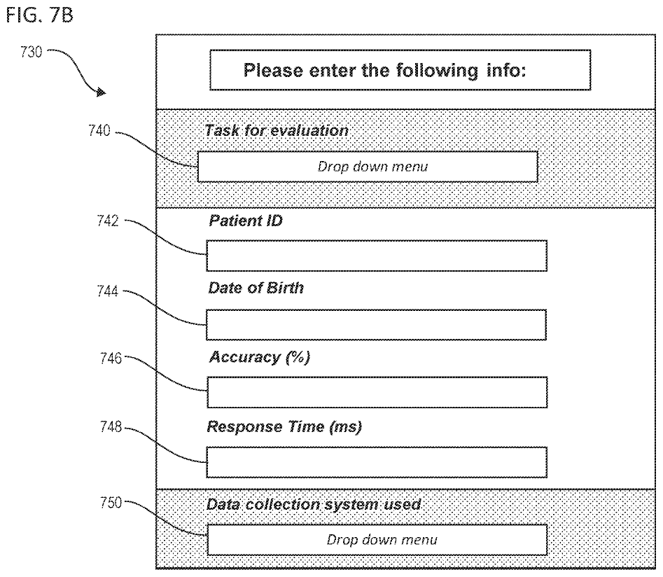

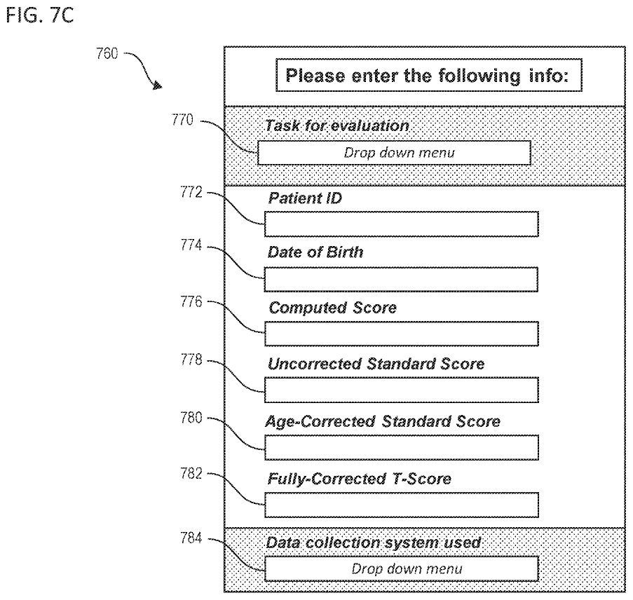

Patient specific information is collected by either a physician directly or through an interface displayed to the patient on a mobile device. For example, demographic data may be collected for purposes of evaluating the effects of Type II diabetes on cognitive abilities via the example system. FIG. 7A is an example patient information interface 700 that allows the collection of patient data for using the example system. The example patient information interface 700 is designed for collecting demographic input data for evaluating a behavioral task input test. In this example, the interface 700 includes a patient ID field 710, a date of birth field 712, a sex field 714, a menopausal status field 716, and a menopausal age field 718. A recent test result data window 720 allows entry of common measurements such as glycated hemoglobin (A1c), body mass index (BMI), and total cholesterol. A task for evaluation drop down menu 732 allows a clinician to select a test to be administered. Thus, an example menu may include options for a stimulation test such as a motor function test, a memory test, tests from the NIH Toolbox, or other tests of brain functions.

A second interface 730 in FIG. 7B collects result data for performance of a user of a memory-based task (e.g., N-back). The interface 730 would be displayed after the input of patient information in the interface 700 in FIG. 7A in response to the selection of a specific memory-based test. The interface 730 includes a task for evaluation drop down menu 740 that allows a clinician to select a test to be administered. The interface 730 includes a patient ID field 742, a date of birth field 744, an accuracy field 746, and a response time field 748. The accuracy data and response time data are recorded from the performance of the memory based task and the application populates the accuracy field 746 and the response time field 748 with the recorded results. A data collection system drop down menu 750 allows a clinician to choose the test. The behavioral task data from the interface 730 is combined with data processed from the signals sensed by the wearable sensor device 110, and patient intake data (e.g., sex, menopausal status, BMI, A1c, cholesterol, etc.) from the interface 700. The data is evaluated via logistic regression to determine clinical status (final output to clinical in terms of mild cognitive impairment (MCI) diagnosis). The example system and logistical regression may also be refined to diagnose specific types of dementia (e.g., vascular dementia vs. Alzheimer's disease).

FIG. 7C shows an example interface 760 that collects result data for performance of a user of a data for behavior task test such as the NIH Toolbox Pattern Comparison Processing Speed Test age 7+. The interface 760 would be displayed after the input of patient information in the interface 700 in FIG. 7A and in response to the selection of the NIH Toolbox test in the interface 700 in FIG. 7A. The interface 760 includes a task for evaluation drop down menu 770 that allows a clinician to select a test to be administered. The interface 770 includes a patient ID field 772, a date of birth field 774, a computed score field 776, an uncorrected standard score field 778, an age corrected standard score field 780, and a fully corrected T-score field 782. The scoring data is recorded from the performance of the behavior test and the fields 776, 778, 780, and 782 are populated by the application based on the test results. A data collection system drop down menu 784 allows a clinician to choose another test. Behavioral tasks used by the example system may also include standard testing via the NIH Toolbox. Independent of behavioral data source, the behavioral data from the interface 760 will be combined with and patient intake data (e.g., sex, menopausal status, BMI, A1c, cholesterol, etc.) from the interface 700. The data is evaluated via logistic regression to determine clinical status (final output to clinical in terms of MCI diagnosis).

For example, as a result of the behavior and memory tests, changes in hemoglobin concentrations may be detected in regions of interest such as dPFC, S1, M1, and supramarginal gyrus regions of interest, by changes in the optical signals indicating changes in such concentrations. The changes are correlated with the inputs from the patient taking the test and the patient specific demographic data. A determination of cognitive impairment may be made based on a comparison of the changes or lack of changes with a baseline of expected changes in the regions of interest.

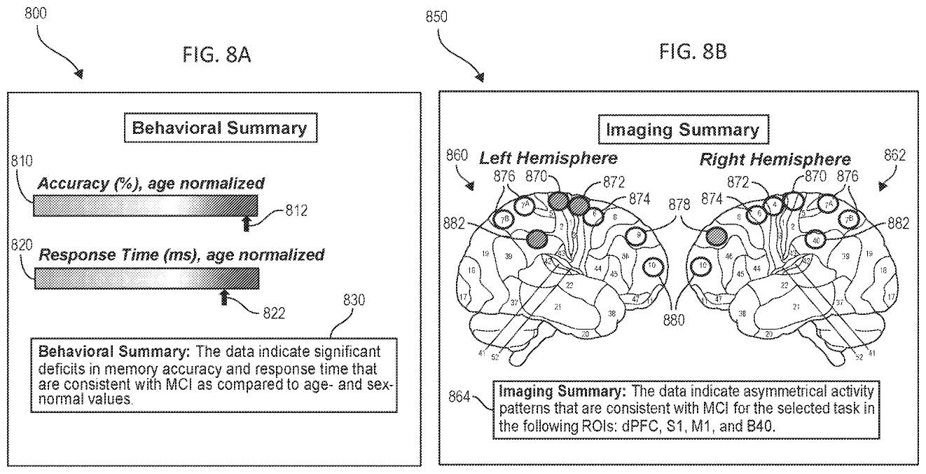

FIG. 8A shows an example behavior summary output interface 800 generated by the example analysis system. The behavior summary output interface 800 summarizes the performance of the patient on the selected stimulation task (e.g., a memory based task test). A first bar 810 shows an accuracy score via an arrow 812. In this example, the left side of the bar 810 may be color coded as green indicating a high accuracy. The left side of the bar 810 may be color coded as red indicating a low accuracy. The bar 810 may be colored to transition from green to red, reflecting the percentage accuracy. The score is normalized for the age of the patient.

A second bar 820 shows a response time score via an arrow 822. The score is also normalized for the age of the patient. The second bar 820 may also be color coded so low response times on the left of the bar 820 have a green color while high response times on the right of the bar 820 may have a red color. The resulting analysis is shown in a result summary box 830. In this example, the summary is based on statistical comparison with normative data from a database of healthy patients. A conclusion is generated from selecting descriptions of the comparisons. In this example, the summary is that there are significant deficits in memory accuracy and response time that is consistent with MCI.

FIG. 8B shows an example imaging summary output interface 850 that may be generated from the data collected by the wearable sensor device 110 in FIG. 1. The output interface includes a left brain map 860, a right brain map 862, and a summary box 864. The left brain map 860 and right brain map 862 have regions of interest highlighted to assist in analysis. In this example, the regions of interest in each of the brain maps 860 and 862 include a circle 870 around the primary sensory cortex (S1), a circle 872 around the primary motor cortex (M1), a circle 874 around the supplemental motor area (SMA), a circle 876 around the visuomotor coordination area, a circle 878 around the dorsolateral pre-frontal cortex (dPFC), a circle 880 around the anterior pre-frontal cortex (aPFC), and a circle 882 around the supramarginal gyrus.

The information in FIGS. 8A-8B may be used for analysis of mild cognitive impairment (MCI) in a patient. As shown in FIG. 8B, oxygenated hemoglobin (HbO), deoxygenated hemoglobin (HbR), and total hemoglobin (HbT) levels changed in certain areas, which are highlighted in the brain maps 860 and 862 by the application based on the analysis of the optical data from the optical sensors corresponding to the areas. In this example, the circles 870, 872 and 882 representing the primary sensory cortex (S1), the primary motor cortex (MI), and the supramarginal gyrus are highlighted in the map brain 860. The circle 878 representing the dorsolateral pre-frontal cortex (dPFC) is highlighted in the brain map 862. The highlighted circles 870, 872, 878, and 882 indicate that changes were detected in the dPFC, S1, M1, and supramarginal gyrus regions of interest in response to the memory test based on the collection of optical signals from paired optical emitters and detectors.

In this example, the regions of interest with HbO, HbR, and/or HbT levels with p-values of less than 0.05 are highlighted (e.g., being shaded red) in the maps 860 and 862. Since ROI values were p<0.05 in these ROI, a comparison to age-and sex-matched healthy control data is made in the software. The software determines that these ROI activations were not consistent with values exhibited by age-and sex-matched healthy controls. The imaging summary 864 is generated by the software and provides the finding of the MCI based on the highlighted regions of interest.

Thus, in this example, a determination of early dementia is indicated by comparative analysis to healthy age-and sex-matched controls and logistical regression techniques within the software routine. The severity of dementia is also indicated by comparative analysis to healthy age-and sex-matched controls and logistical regression techniques within the software system.

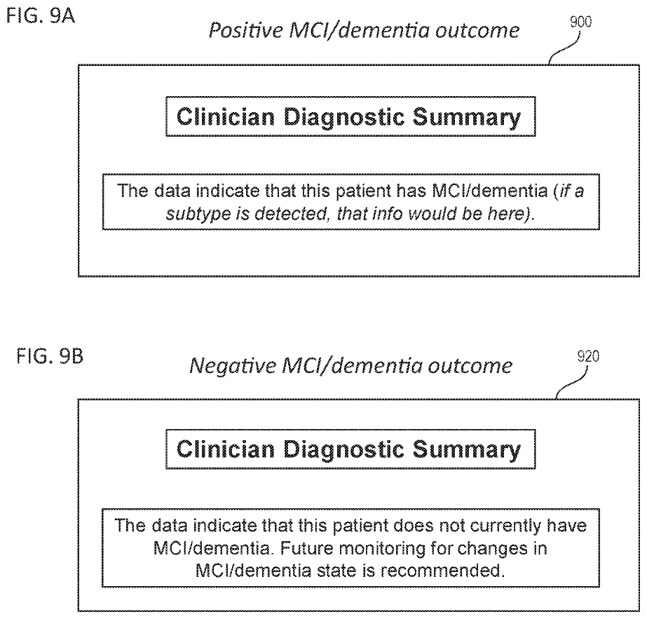

FIG. 9A shows an example screen image 900 of a positive outcome of a dementia outcome. The screen image 900 may be displayed to the workstation display 116 to the clinician. The positive outcome may include information such as the subtype detected. FIG. 9B shows an example screen image 920 of a negative outcome of detection of MCI. Additional recommendations may be included in the output.

Experiments were conducted to show that altered hemodynamic function of the cortex leads to type II diabetes mellitus (DM) complications including cognitive and sensorimotor impairments. In particular, postmenopausal women likely experience significant deterioration of both hemodynamic function and overt behaviors (e.g., cognitive function) given their disproportionate risk of cardiovascular complications as compared to men with DM and individuals without DM. The experiments evaluated changes in cortical oxygenation indices of postmenopausal women both with and without DM during memory-based cognitive single-and dual-tasks via the example functional near infrared spectroscopy based detection and testing method.

The experiments were conducted to examine whether between-group differences in cognitive function, impaired memory/recall would occur in the DM group (Hypothesis #1). Concurrent with impaired cognitive function, the experiments were to determine expected between-group differences in cortical oxygenation indices of oxygenated hemoglobin (HbO) and deoxygenated/reduced hemoglobin (HbR) (Hypothesis #2) across regions of the cortex involving memory and sensorimotor function during tasks involving cognitive components. To examine the two hypotheses, cortical hemodynamic activity was measured with the example functional near infrared spectroscopy (fNIRS) based wearable sensor device during performance of cognitive tasks by the test subjects to evaluate the relationship between cortical hemodynamic activity and cognitive function in persons with DM versus controls.

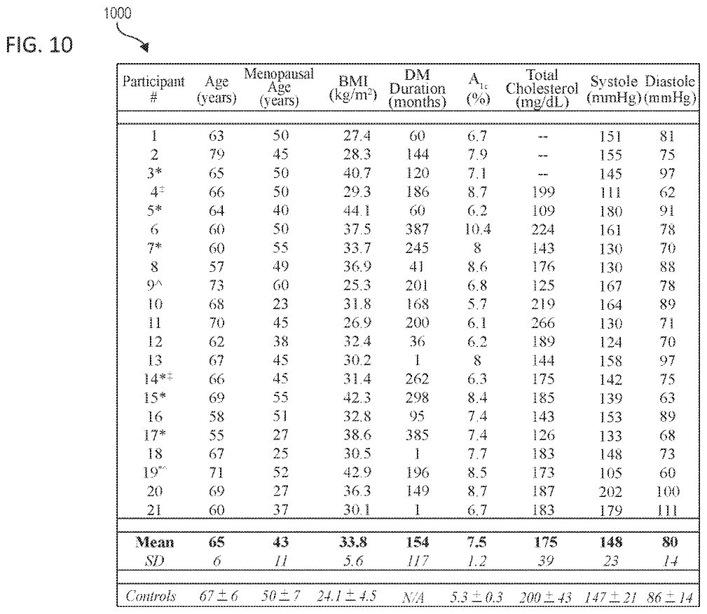

Twenty-one postmenopausal women with DM and twenty-one age-and sex-matched healthy controls volunteered to participate in the case control study. FIG. 10 is a table 1000 that lists the demographics for the participants in the experiment. Handedness was assessed by the Edinburgh Inventory, ranging from a laterality quotient (LQ) of −100 (strong left-handedness) to +100 (strong right-handedness). Participants had an LQ average of +88 and had no previous history of trauma to the upper limbs. Both the DM group and control group included women from self-identified underrepresented racial and ethnic minority groups (n=24/42 (57%)). Study participants were excluded if they reported a history of neurological and/or musculoskeletal disorders (Parkinson disease, Huntington's disease, polio, multiple sclerosis, stroke, traumatic brain injury, carpal tunnel syndrome, rheumatoid arthritis, Monoclonal Gammopathy of Undetermined Significance (MGUS), Paraproteinaemic Demyelinating Neuropathy (PDN), Myasthenia Gravis), a history of amputation, a history of major surgical intervention to the upper extremity, or hereditary or compression neuropathies. In accordance with the Declaration of Helsinki, participants provided informed consent according to the regulations established by the Institutional Review Board at the University of Houston (protocol #15615-01). Data collection processes failed on five participants (e.g., a reliable fNIRS signal was not detected (control participants #2, #7, and #10; DM participants #9 and #19)). Data from those participants were excluded from fNIRS analyses but not behavioral data for completeness of reporting.

Blood pressure, cholesterol, and glycated hemoglobin (A1c) values were assessed for all study participants onsite at the onset of each session. Cholesterol and A1c values were assessed using a commercially available point of care evaluation kit (Cardiocheck+ and A1c Now+ kits, PTS Diagnostics, Indianapolis, IN, USA). Blood pressure was measured using a commercially available device (an Omron Intellisense 10 series Blood Pressure Monitor, Model BP785, Bannockburn, IL, USA). The presence of peripheral neuropathy (PN status) was determined by abnormalities on either clinical examination or EMG/NCV testing (per physician). A brief menopause questionnaire was also administered regarding several aspects of menopausal characteristics (e.g., age at onset of menopause, hormone replacement therapy history, etc.). All study participants declared themselves to be postmenopausal; with 11 participants claiming a history of hormone replacement therapy (5 with a history of Prempro use). Of the 11 participants with a history of hormone replacement therapy, 4 were in the control group and 7 were in the DM group.

Cognitive function of each participant was screened using the Montreal Cognitive Assessment (MoCA). This is a brief examination of the cognitive domains: attention and concentration, executive functions, working memory/recall, language, visuo-constructional skills, conceptual thinking, calculations, and orientation. The number of years of patient education is accounted for within the MoCA scoring structure. This evaluation was performed prior to placement of the wearable device with the functional near infrared spectroscopy sensors.

A series of tasks were performed by the test subject. A first task related to working memory (N-Back) evaluation (single-task). Working memory of each participant was probed using the working memory (N-back) evaluation while wearing the example fNIRS sensor device. Working memory was assessed while participants were seated in a quiet location. This test required participants to repeat the “Nth” word back in a list of random words presented as auditory stimuli. The difficulty level was controlled by requiring participants to remember words further back in the series. Three conditions of the N-back task were assigned to each subject (easiest to most difficult: 0-, 1-, and 2-back conditions) in a block randomized manner. Participants wore a headset with headphone and microphone capabilities (Plantronics Inc., Santa Cruz, California), through which they heard a randomized sequence of words via audio provided by E-prime 2.0 (Psychology Software Tools, Inc., Sharpsburg, PA). The software program generated randomized words through the headphones at an interval of 2s per word. Participants were instructed to verbally repeat the words into the headset in the correct sequence for a task duration of 30s. The rate of correct responses and verbal reaction time were recorded by the E-prime software and extracted to evaluate performance. Three trials were collected in each of the N-back conditions. N-back conditions were block randomized across all participants.

A second related to working memory (N-Back)+motor task evaluation (dual-task). The working memory function was probed at a baseline (single-task) as well as during motor function evaluations (dual-task). All single-tasks occurred prior to dual-tasks to avoid subject confusion. Each subject was asked to perform a series of working memory+motor task (dual-task) interleaved by 30s periods of rest.

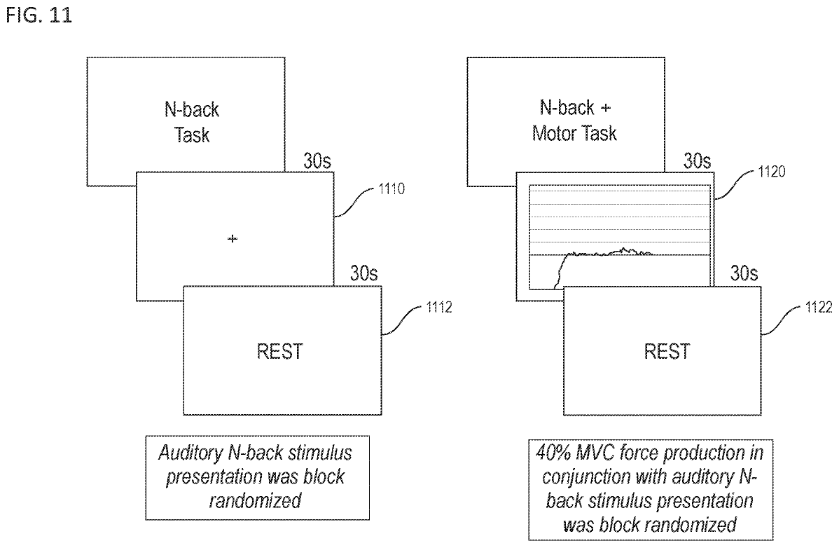

FIG. 11 shows experimental stimuli used during the N-back single task test and the N-back+motor performance (dual-task) test. In the N-back single task block, subjects viewed an image 1110 with a fixation cross followed by an image with a rest instruction 1112. In the N-back+motor performance dual-task block, they viewed real-time feedback as shown in an image 1120 on their force production during N-back (dual-task) blocks, followed by an image with a rest instruction 1122. The order of N-back presentation was block randomized within each testing type. N-back single task tasks always occurred prior to N-back+motor performance dual-tasks.

Presentation of visual stimuli, timing, and synchronization TTL signals were controlled via E-prime 2.0 (Psychology Software Tools, Inc., Sharpsburg, PA). Three trials were collected in each of the N-back conditions for dual-task evaluation. N-back conditions were block randomized across all participants in dual-task conditions.

During the working memory +motor task, participants used a precision pinch grip to exert an isometric force against a set of force transducers. Participants were instructed to match their pinch force to the target force line in the image 1120 as accurately as possible. Two different force levels were tested for the dominant (right) hand (15% MVC and 40% MVC). Three trials of 30s each, were performed with at 30s of rest/washout periods between each block. Force level order (15% or 40% MVC) was block randomized.

The motor task involved using digits 1 and 2 in a precision pinch grip to produce a constant level of pinch force, with feedback from a computer screen. All forces and moments of force produced were recorded simultaneously using 2 identical 6-component force-moment transducers (Nano-25 transducers; ATI Industrial Automation, Garner, NC, USA).

Cortical hemodynamics were measured with a continuous-wave functional near infrared spectroscopy instrument (NIRScout, NIRx Technologies, Glen Head, NY, USA) via 16 optical emitters and 16 optical detectors in an example wearable device in FIG. 1. Each optical emitter consisted in a dual-wavelength LED (central wavelengths: 760 nm and 850 nm) directly coupled to the scalp, while each detector was a silicon photodiode collecting backscattered light from the scalp via an optical fiber. The geometrical layout of optical emitters and detectors (collectively referred to as optodes) is shown in FIG. 5C, alongside the corresponding sensitivity map of the optical probing on the cerebral cortex in FIG. 5D. The sensitivity map was estimated with Monte Carlo-based simulation of photon migration in Atlas Viewer. Reproducibility of placement was ensured by fitting the standard 10-10 headset (EasyCap, Germany) with reference to anatomical landmarks (nasion Nz, inion Iz, vertex Cz, preauricular points LPA and RPA), to achieve an optode landing according to the layout depicted in FIG. 5C. The spatial location of all optodes was digitized and registered such position to a scalp-brain atlas (Colin 27) to ensure placement accuracy within reasonable range (10 mm from standard EEG labels). Regarding the association between optode placements and cortical regions, cortical areas interrogated by each group of optical channels (ROIs) were inferred from the sensitivity map projected onto a Colin 27 model computed with photon migration simulations using Atlas Viewer. Although FIG. 5C shows the sensitivity map of the entire probe, the projections of each ROI were displayed separately and denoted cortical regions accordingly. In this example, there are 16 pairs of optical emitters and optical detectors. Certain channels included optical emitters or detectors used for other channels. The geometrical distance between optode emitter and detector pairings ranged from 26 to 37 mm, ensuring the interrogation of the cerebral cortex in all optical channels. Proper scalp-optode coupling was ensured by using the PHOEBE toolbox. This configuration resulted in taking reading from 28 optical channels (i.e., emitter-detector pairings) from 52 possible channels. The 28 optical channels interrogated the prefrontal, motor, and somatosensory cortices bilaterally.

Raw optical signals were collected continuously throughout the N-back portions of the experiment at the frequency of 3.91 Hz from all channels at both wavelengths, and were subsequently converted to optical density (i.e., logarithm of the raw intensity) and then to concentration changes of oxygenated (HbO) and deoxygenated hemoglobin (HbR) compared to a zeroed baseline according to the modified Beer-Lambert Law. For each channel, HbO and HbR measurements were analyzed separately with a general linear model approach that estimated the scalar weight coefficient (a.k.a., beta weight) of the canonical hemodynamic response that best fitted the measured hemodynamic response. Particular preprocessing steps were not applied to the fNIRS data, since autoregressive pre-whitening approach using iteratively reweighted least-squares (AR-IRLS) dealth with data outliers produced by motion artifacts and extracerebral and physiological responses. For each subject, channels were considered as hemodynamically active if their weight coefficient was statistically different from zero at the significance level of 5%.

At the group level, a mixed linear model was used to estimate the weighting coefficient of all channels to determine which of them were hemodynamically active at a statistically significant level. The interaction between the experimental condition (N-back condition) and the group (DM vs. control) was considered as the fixed effect contributing to the weight coefficient, while the magnitude of the coefficient of individual subjects was considered as a random effect.

Optical channels were grouped into ten bilateral (right and left) regions of interest (ROIs), namely the prefrontal cortex (PFC), supplementary motor area (SMA), primary motor cortex (M1), primary sensory cortex (S1), and Brodmann Area 40 (B40) as depicted in FIG. 5C. Individual-level ROI-level statistics (weight coefficient, t-value, p-value) were computed. Positive HbO values and negative HbR values each indicate cortical activity, respectively. Some ROIs did not produce significant t-scores in HbO or HbR. This data are shown as zeroes in mean and standard error (SE) values in the results.

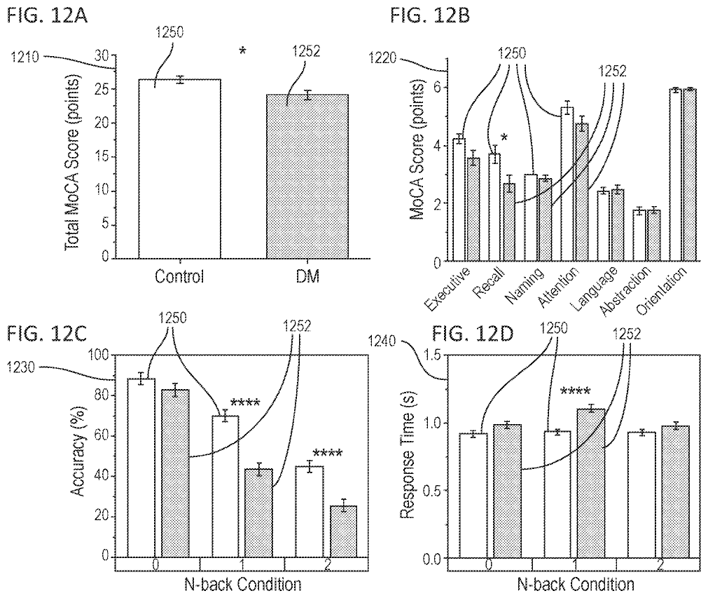

The data is presented as means±SE. For HbO and HbR, statistically significant individual-level ROI t-scores were compared between Groups using mixed model analyses of covariance (ANCOVAs) via SPSS 25 (IBM Corporation, Armonk, NY, USA). Between-subject primary factors were Group (two levels: DM vs. controls). Within-subject factors included Hemisphere (two levels for the cortex: left and right) and ROI (five levels: 1=PFC, 2=SMA, 3=M1, 4=S1, and 5=B40). For N-back data, main factors included: Group, Task Type (two levels: one level each for single-and dual-tasks), and Condition (three levels: 0-back, 1-back, and 2-back). Evaluation of health state covariates was performed to control for health state variability both within and across the two sample groups. Covariates were selected via Automatic Linear Modeling (ALM) using forward stepwise selection functions in SPSS. ALM was utilized to reduce the potential for expectation biases that may occur when hand-selecting potential statistical models. In the event of significant covariates determined via ALM and ANCOVA, follow-up correlation analyses were performed between the health state or performance covariate and the measured behavior. ANCOVAs included health state covariates of: A1c, systolic and diastolic blood pressures, total cholesterol, high-density lipoprotein (HDL) cholesterol, disease duration, menopausal age, body mass index (BMI), PN status (via indicator variable), history of hormone replacement therapy (via indicator variable), history of treatment with Prempro (conjugated estrogens/medroxyprogesterone acetate; via indicator variable), and working memory performance variables of response time and accuracy (in HbO and HbR analyses). Specific attention to use of Prempro was warranted as long-term use of Prempro is associated with development of cardiovascular disease and potential cognitive complications. In multiple comparison situations, Bonferroni corrected posthocs were used. Significant differences are denoted by the following in the following results in FIGS. 12-14: * at p<0.05, ** at p<0.001, *** at p<0.005, and **** at p<0.001.

The results for the Montreal Cognitive Assessment (MoCA) cognitive tests are shown in the graphs 1210, 1220, 1230 and 1240 in FIG. 12. The graphs 1210, 1220, 1230, and 1240 plot Group mean and standard error (SE) for MoCA and working memory data. A set of bars 1250 indicate data from the control group, and a set of bars 1252 indicate data from the DM group in each of the graphs 1210, 1220, 1230 and 1240. Significant differences between Groups at p<0.05 (*) and p<0.001 (***) are shown. The graph 1210 shows Total MoCA scores from the experiment participants. The graph 1220 shows Domain specific MoCA scores. The graph 1230 shows correct response rates (accuracy) in N-back evaluations. The graph 1240 shows response times in N-back evaluations.

Via ALM, the MoCA data show a significant Group difference (F1,42=6.45, p<0.05) in which the average total MoCA scores were lower in the DM group as compared to controls. Further analyses of the individual MoCA domains indicated Group differences in working memory/recall (F1,19=7.27, p<0.05), such that working memory/recall scores in the DM group were lower as compared to controls.

Differences between single-and dual-task accuracy rates were not found via ALM; subsequent analyses of N-back data were performed collapsed across both single-and dual-task conditions. Significant Group differences in N-back accuracy were found (F1, 230=46.73, p<0.001); such that the DM group was less accurate than controls as shown in the graph 1230. Condition (F2, 230=142.61, p<0.001) and Group x Condition (F2, 230=6.29, p<0.005) effects were found such that accuracy declined as the Condition became more difficult; however, the decline in accuracy was more dramatic in the DM group as shown in the graph 1230. When health state covariates were included in statistical analyses, the Condition (F2,123=170.39, p<0.001) effect remained significant. However, health state covariates of Total Cholesterol (F1,123=9.95, p<0.005), Menopausal Age (F1,123=14.47, p<0.001), and Prempro Use (F1,123=13.86, p<0.001) replaced the Group effect. These health state covariates were positively correlated with accuracy (Total Cholesterol: r264=0.277, p<0.001; Menopausal Age: r252=0.219, p<0.001; Prempro Use: r252=0.137, p<0.05).

In relation to Working Memory (N-Back) Evaluations, differences between single-and dual-task response times were not found via ALM; subsequent analyses of N-back data were performed collapsed across both single-and dual-task conditions. Group differences in N-back response times were found (F1, 217=21.20, p<0.001); such that the DM group had longer response times than controls as shown in the graph 1240. Significant Condition (F2, 217=4.72, p<0.05) and Group x Condition (F2, 217=3.44, p<0.05) effects were found such that response times were generally flat in the control Group but were significantly higher in the 1-back condition for DM group as compared to all other Conditions as shown in the graph 1240. When health state covariates were included in statistical analyses, the main effects of Group and Condition disappeared. Instead, Total Cholesterol (F1,90=16.92, p<0.001) dominated the model and was negatively correlated with response time (r240=−0.257, p<0.001).

For Cortical Hemodynamic responses during Working Memory (N-Back) evaluation, ALM analyses indicated significant differences in Task in the HbO data, but not the HbR data. In the following, the HbO data is presented first with results presented in the single-task separate from the dual-task. Afterwards, the HbR data is presented collapsed across Task, as Task was not found to be a significant factor for HbR.

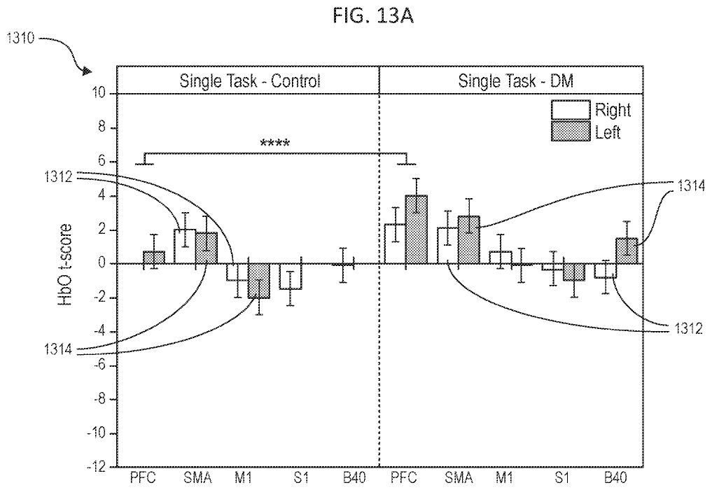

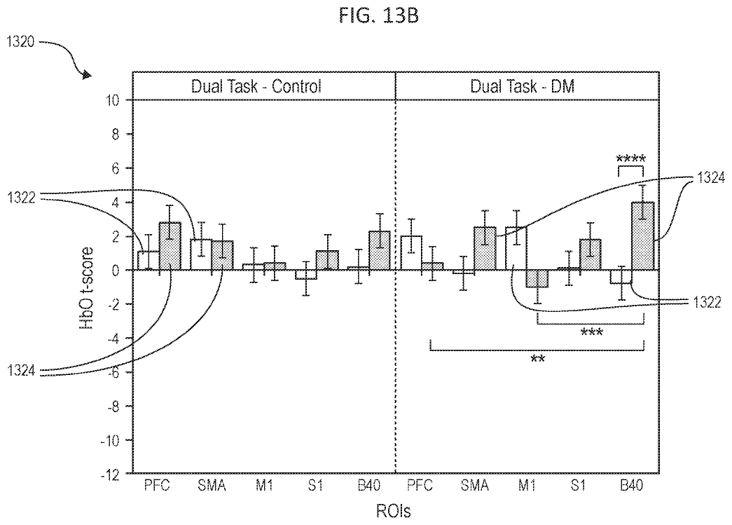

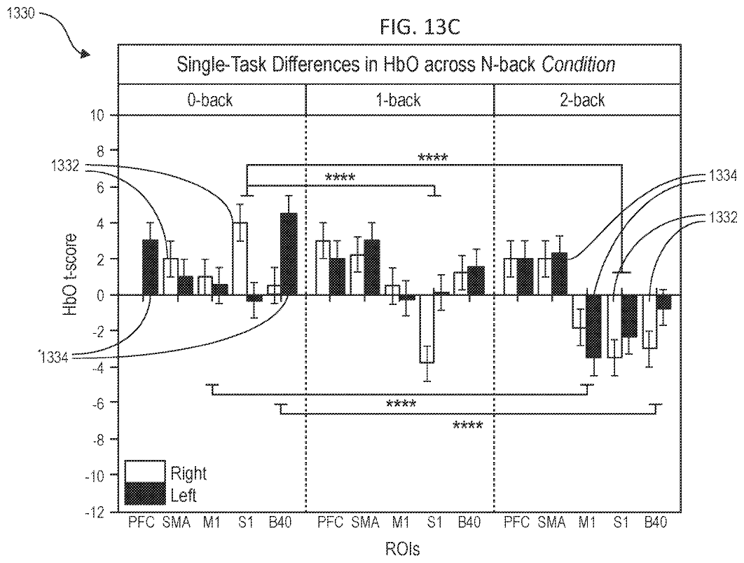

FIG. 13A shows a graph 1310 of fNIRS t-scores for HbO during single-task evaluation for each Group, depicted by ROI and Hemisphere. A set of bars 1312 indicate right hemisphere and a set of bars 1314 indicate left hemisphere. FIG. 13B shows a graph 1320 of fNIRS t-scores for HbO during dual-task evaluation for each Group, depicted by ROI and Hemisphere. Mean and standard error (SE) values are shown. Significant at p<0.01 (**) , p<0.005 (***) , p<0.001 (****) are shown. A set of bars 1322 indicate right hemisphere and a set of bars 1324 indicate left hemisphere. FIG. 13C shows a graph 1330 of fNIRS t-scores for HbO during N-back single-task evaluations (0-, 1-, and 2-back Conditions), depicted by ROI and Hemisphere. Data are averaged across Group. Mean and standard error (SE) values are shown in the graph 1330. Significant differences between N-back Conditions at p<0.001 (****) are shown. A set of bars 1332 indicate right hemisphere and a set of bars 1334 indicate left hemisphere.

During the single-task working memory evaluation, significant effects of Group (F1,76=4.07, p<0.05), ROI (F4,76=5.40, p<0.001), and Condition (F2,76=5.77, p<0.001) were found in HbO t-scores via ALM. Overall, the data show significantly larger average HbO t-scores in the DM Group as compared to controls; this is particularly noticeable in PFC (between Group differences are denoted in the graph 1310). As the N-back Condition became more difficult (0-back vs. 2-back), HbO t-scores decreased significantly on average across ROIs except for PFC and SMA, denoted in the graph 1330. HbO t-scores in PFC were significantly different from S1 and M1 as N-back Condition difficulty increased (shown in the graph 1330), supported by a near significant interaction in Condition x ROI (F8,76=1.77, p=0.096). No health state covariates were found impact to HbO t-scores in the single-task condition.

During the dual-task working memory evaluation, a significant interaction effect in HbO of Group x Side x ROI (F13,170=1.971, p<0.05), shown in the graph 1320, was found when response time and accuracy were included as covariates within the statistical model via ALM. Posthoc analysis of this data show significantly higher HbO t-scores by the DM Group in the left hemisphere in the dual-task (most notably in B40 as compared to PFC and M1), denoted in the graph 1320. No health state covariates were found impact to HbO t-scores in the dual-task condition.

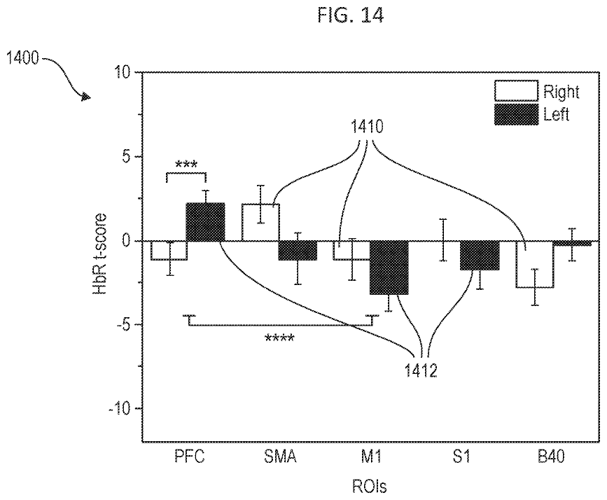

FIG. 14 is a graph 1400 that shows NIRS t-scores for HbR collapsed across all tasks and conditions, depicted by ROI and hemisphere. Mean and standard error (SE) values are shown. Significant differences at p<0.005 (***) and p<0.001 (****) are shown. A set of bars 1410 indicate right hemisphere and a set of bars 1412 indicate left hemisphere. With respect to HbR data, collapsed across task, a significant effect of ROI (F4,187=2.60, p<0.05) was found along with a significant Side x ROI interaction (F4,187=3.93, p<0.005) via ALM, as indicated in the graph 1400. HbR t-scores showed significant asymmetry in the PFC region, as well as significant differences between PFC and M1 activation in both hemispheres (supported by posthoc testing). No health state covariates were found impact HbR t-scores.

The purpose of the experiments was to evaluate changes in cortical oxygenation indices of postmenopausal women postmenopausal women both with and without DM during cognitive tasks. The result data shows that cognitive impairment in memory/recall was observed in postmenopausal women with DM as compared to controls. Impaired memory function appeared as reduced accuracy and did not differ if the task was performed alone or coupled with a simultaneous motor task. Further, HbO values differed between groups during memory/recall tasks; in some ROIs, differences in HbO were magnified in the DM group, suggesting changes in memory activation patterns with increased functional activity of non-PFC regions in PwDM. There is an influence of poor health state and earlier menopausal age on poor memory function; however, no influence of health state was found to impact HbO or HbR.

The data indicates a significant difference in the use of HbO concurrent with impaired memory function, such that the DM group exhibited differences in PFC HbO activity during dual-tasks and dedifferentiation of functional brain activity across remaining ROIs as compared to controls. Functional activity changes concurrent with deficits in working memory in the DM group indicate a functional root for memory deficits in persons with DM that is linked to HbO. This data indicates that memory deficits are a result of a problem with the hemodynamic response, which leads to behavioral deficits in DM. This supports use of behavioral monitoring along with fNIRS through the example system to detect early MCI development since techniques such as fMRI rely on the paramagnetism of HbR, thereby not fully measuring cortical hemodynamic activity which involves both HbO and HbR. Increased HbO use during dual-tasks is notable in the DM group, as HbO use is not indicated by other functional imaging techniques such as fMRI. By its nature, HbO is diamagnetic and not attracted to any magnetic field. The example fNIRS detection system offers better insight into cortical activity using a more inclusive approach that may better reflect early markers of MCI during realistic tasks similar to activities of daily living in populations at high risk of developing dementia. Aberrations in cortical activity may be a potential biomarker for tracking changes in cognitive decline in DM. The aberrations in cortical activity may be detected using wearable technology such as the example fNIRS wearable sensor device. This detection may be achieved ahead of development of dementias such as Alzheimer's disease. Detection of cortical activity differences via fNIRS provides an inclusive approach and expands monitoring eligibility for persons with implanted devices (e.g., stents, pacemakers, etc.) that cannot use known detectors such as fMRIs.

Significantly different use of HbO in the cortex in DM may indicate reduced bioavailability of oxygen in DM; consistent with evidence of behavioral impairment in DM. However, the change in HbO use in the DM group during dual-tasks was not accompanied by improved memory, as accuracy and response time did not differ in either group in the single-and dual-task conditions. DM is associated with increased hemoglobin-oxygen affinity, which is responsible for lower oxygen delivery rates to tissue. DM is also associated with impaired hyperemic response, endothelial dysfunction, and microvascular dysfunction. However, the increased use of HbO in the DM group within the current data set indicate that increased hemoglobin-oxygen affinity does not contribute to the observed memory deficits; rather the impairment in vascular function drives memory deficits in DM.

The DM group exhibited significant bilateral PFC activation via HbO in dual-tasks as compared to controls, despite memory error rates not improving with increased PFC activity. These activity differences co-occurred with activation of non-PFC cortical areas involved in movement, priming for movement, phonological processing, and emotional responses (M1, SMA, and B40 respectively). This DM-specific shift in HbO use is a novel finding that cannot be detected by current fMRI techniques. An increase of HbO along with higher HbO values in other measured ROIs suggests distributed cortical HbO activity in DM to compensate for memory deficits. This change in HbO was not accompanied by group differences in HbR use, suggesting that altered HbO use across the cortex is the driver of memory deficits in DM. Changes in HbO in the DM group are supported by evidence of increased HbO use in the primary visual cortex in PwDM during visual stimulation, and may suggest an increased sympathetic drive in the autonomic nervous system in postmenopausal women. These differences may also suggest potential advanced aging of the brain via cortical dedifferentiation in PwDM beyond what is to be expected with healthy aging.

Changes in PFC activity in HbO use are consistent with reports of hypothalamic-pituitary-adrenal axis (HPA) dysfunction, insulin signaling aberrations, and pathological changes in hippocampal functions all associated with DM. The PFC-hippocampus interaction is known to be important for episodic memory. Metabolic disruption of the PFC-hippocampus via endocrine dysfunction in DM impacts memory and behavior. Aberrations in PFC activity spurred by changes in the HPA axis in DM are consistent with impaired stress coping ability and symptoms of cognitive decline consistent with impaired memory symptoms.

Consistent with past studies, working memory data was impacted by menopausal age and use of specific hormone replacement therapies (HRT). Increased menopausal age (resulting in a shorter time between menopause and participation in the current study) and Prempro use were associated with higher working memory accuracy. Menopausal age was significantly different between the DM (43±11 years) and control (50±7 years) groups (t40=2.85, p<0.05); however, DM-related deficits in accuracy persisted once menopausal age was considered in the statistical models. In contrast, no significant influences of menopause or HRT were found on cortical activity. The lack of a specific impact of menopausal age on cortical hemodynamic response during memory tasks is an intriguing outcome, as menopause is associated with impaired hemodynamic responses of the cortex and skeletal muscle during sensorimotor tasks. There is some evidence that HRT improves hemodynamic responses in postmenopausal females.

Although the examples related to detection of cognitive decline as a possible result of diabetes, the example cognitive evaluation system may be used to detect cognitive decline related to other ailments such as genetic conditions, cardiovascular disease, metabolic disorders, addiction, or as the result of medical treatment (e.g., chemotherapy). In addition, the changes in hemodynamic responses detected by the wearable sensor may have other applications such as monitoring for healthy function in everyday environments or for health and safety monitoring in extreme environments (e.g., high altitude or space).

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A system to evaluate brain activity, comprising:
a head mounted sensor operable to be worn by a user, the head mounted sensor including an optical sensor for measuring an optical response from a region of interest in the brain of the user;
a user input interface receiving a user test input from a test device executing a stimulation test to the user; and
a controller coupled to the sensor device and the user input interface, the controller collecting optical response data from the optical sensor and user test input from the user input interface, the controller operable to:
determine a hemoglobin level in the region of interest based on the optical response data;
correlate the determined hemoglobin level in the region of interest with the user test input in response to the stimulation test; and
evaluate brain activity based on the determined hemoglobin level.

2. The system of claim 1, wherein the head mounted sensor includes an underside, and wherein the underside includes teeth to interface with the hair of the user to stabilize the head mounted sensor when worn by the user.

3. The system of claim 1, wherein the head mounted sensor further includes a curved support structure conforming to the head of the user and lateral wings coupled to the support structure.

4. The system of claim 1, wherein the optical sensor is positioned in proximity to the region of interest of the brain of the user when the sensor is worn by the user.

5. The system of claim 4, wherein the region of interest of the brain is at least one of an anterior pre-frontal cortex (aPFC); a dorsolateral pre-frontal cortex (dPFC); a supplemental motor area (SMA); a primary motor cortex (M1); a primary sensory cortex; a visuomotor coordination area; or a supramarginal gyrus.

6. The system of claim 1, wherein the input interface is in communication to a mobile device administering the stimulation test to the user, wherein the mobile device includes a transceiver to send the input data to the controller.

7. The system of claim 6, wherein the mobile device executes an application to administer the stimulation test, and wherein the stimulation test is one of a working memory (N-back) evaluation or the Montreal Cognitive Assessment (MoCA).

8. The system of claim 6, wherein the stimulation test is one of a combined memory test and motor function test or a memory test.

9. The system of claim 1, further comprising a display coupled to the controller, the display operable to display the brain activity of the region of interest of the brain of the user based on the collected optical data and an indication of cognitive impairment based on the brain activity.

10. The system of claim 9, wherein the cognitive impairment is dementia.

11. The system of claim 1, wherein the optical sensor is an optode including a dual-wavelength LED emitting light to the region of interest, a silicon photodiode and an optical fiber coupled to the silicon photodiode, wherein the silicon photodiode collects backscattered light from the region of interest and transmits the signal to the controller via the optical fiber.

12. The system of claim 1, wherein the optical response is associated with concentration changes of one of oxygenated (HbO) hemoglobin, deoxygenated hemoglobin (HbR), total hemoglobin, or glycated hemoglobin in the region of interest.

13. A method of determining brain activity in a user, the method comprising:
receiving a user test input from a test device executing a stimulation test to the user;
providing an optical input signal to a region of interest of the brain of the user via a head mounted sensor worn by the user,
collecting optical response data from an optical detector in the region of interest;
determining a hemoglobin level in the region of interest based on the optical response data;
correlating the determined hemoglobin level in the region of interest with the user test input in response to the stimulation test; and
evaluating brain activity based on the determined hemoglobin level.

14. The method of claim 13, wherein the region of interest of the brain is at least one of an anterior pre-frontal cortex (aPFC); a dorsolateral pre-frontal cortex (dPFC); a supplemental motor area (SMA); a primary motor cortex (M1); a primary sensory cortex; a visuomotor coordination area; or a supramarginal gyrus.

15. The method of claim 13, wherein the stimulation test includes one of a working memory (N-back) evaluation, the Montreal Cognitive Assessment (MoCA), a combined memory test and motor function test, or a memory test.

16. The method of claim 13, further comprising displaying brain activity of the region of interest of the brain of the user based on the collected optical data and an indication of cognitive impairment based on the brain activity.

17. The method of claim 13, further comprising determining cognitive impairment from the evaluation of brain activity.

18. The method of claim 13, wherein the optical signal is provided via an optical emitter including a dual-wavelength LED emitting light to the region of interest, and wherein the optical detector is a silicon photodiode and an optical fiber coupled to the silicon photodiode, wherein the silicon photodiode collects backscattered light from the region of interest.

19. The method of claim 13, wherein the optical response is associated with concentration changes of one of oxygenated (HbO) hemoglobin, deoxygenated hemoglobin (HbR), total hemoglobin, or glycated hemoglobin in the region of interest.

20. The method of claim 13, wherein the head mounted sensor includes an underside, and wherein the underside includes teeth to interface with the hair of the user to stabilize the head mounted sensor when worn by the user.

21. The method of claim 13, wherein the head mounted sensor further includes a curved support structure conforming to the head of the user and lateral wings coupled to the support structure.

22. A head mounted device for collecting data for evaluating brain activity in a patient, the device comprising:
a blade structure having a curved shape operable for contact between the forehead and back of the head of a patient;
a plurality of optodes in the blade structure, the plurality of optodes each including a dual-wavelength LED, a silicon photodiode and an optical fiber, wherein each of the plurality of optodes are located in proximity to a respective region of interest of the brain of the patient indicative of brain activity, the LED emitting an input signal to the respective region of interest, the silicon photodiode collecting a response optical signal from the respective region of interest; and
a data interface collecting the response optical signals from the optical fibers of the optodes to determine hemoglobin levels of the respective regions of interest.

23. The head mounted device of claim 22, wherein the blade structure includes an underside configured to contact the head of the patient, the underside including teeth attaching to hair of the patient.

24. The head mounted device of claim 22, further comprising a transceiver to send the collected optical signals to an external device.

25. The head mounted device of claim 23, further comprising a pair of lateral wings extending perpendicularly from the blade structure, the lateral wings contacting the head of the patient to hold the head mounted device in place.
